(19) United States
(12) Patent Application Publication
(10) Pub. No.: US 2008/0195157 A1
(43) Pub. Date: Aug. 14, 2008

(54) Small Fragment Humeral Head Plate

(75) Inventors: Erich Orschler, Rodental (DE); Frank Orschler, Konigsee (DE); Ulrich Finger, Saalfeld (DE); Arndt Ehrhardt, Domfeld (DE)

Correspondence Address: BODNER & O'ROURKE, LLP, 425 BROADHOLLOW ROAD, SUITE 108, MELVILLE, NY, 11747, US

(73) Assignee: Konigsee Implantate und Instrumente zur Osteosynthese GmbH, Konigsee-Aschau (DE)

(21) Appl. No.: 11/795,079

(22) PCT Filed: Dec. 19, 2005
(86) PCT No.: PCT/EP05/13679

§ 371 Date: Mar. 20, 2008

(30) Foreign Application Priority Data
Jan. 13, 2005 (DE) 20 2005 000 508.8

(51) Int. Cl. A61B 17/58; A61B 17/56

(52) U.S. Cl. 606/280; 606/60; 606/102

(57) ABSTRACT

The invention relates to a small fragment humeral head plate which is used to fix, in a stable manner, bone fracture fragments to the humeral head and shaft which is near to the body, including an elongate plate part which is sheet-shaped, in addition to an anatomically shaped head part which is attached thereto and bores which are arranged in the plate part and in the head part, said bores having a conical-shaped thread which is used to receive bone screws which comprise, respectively, a screw head with a complementary conical thread, such that an angle-stable position is produced when screwing. According to the invention, a symmetrically distributed group of bores are embodied in the head part, wherein the axis of symmetry coincides with the longitudinal axis of the plate. The first, external bores on the head part are oriented in relation to each other at an angle of between, approximately 12 degrees-17 degrees, preferably 15 degrees. The second, internal bores on the head part are oriented in relation to each other at an angle which is smaller than the external bores of the head part, preferably at approximately 10 degrees. The first and second bores have an inclined position relative to the external head part in relation to the perpendicular of the longitudinal axis of the plate. The invention also relates to an additional bore which is embodied, preferably, on the longitudinal central axis at a predetermined distance in relation to the proximal end of the plate, whereby the inclined position angle thereof, in the proximal direction, is greater than the first and second bores.

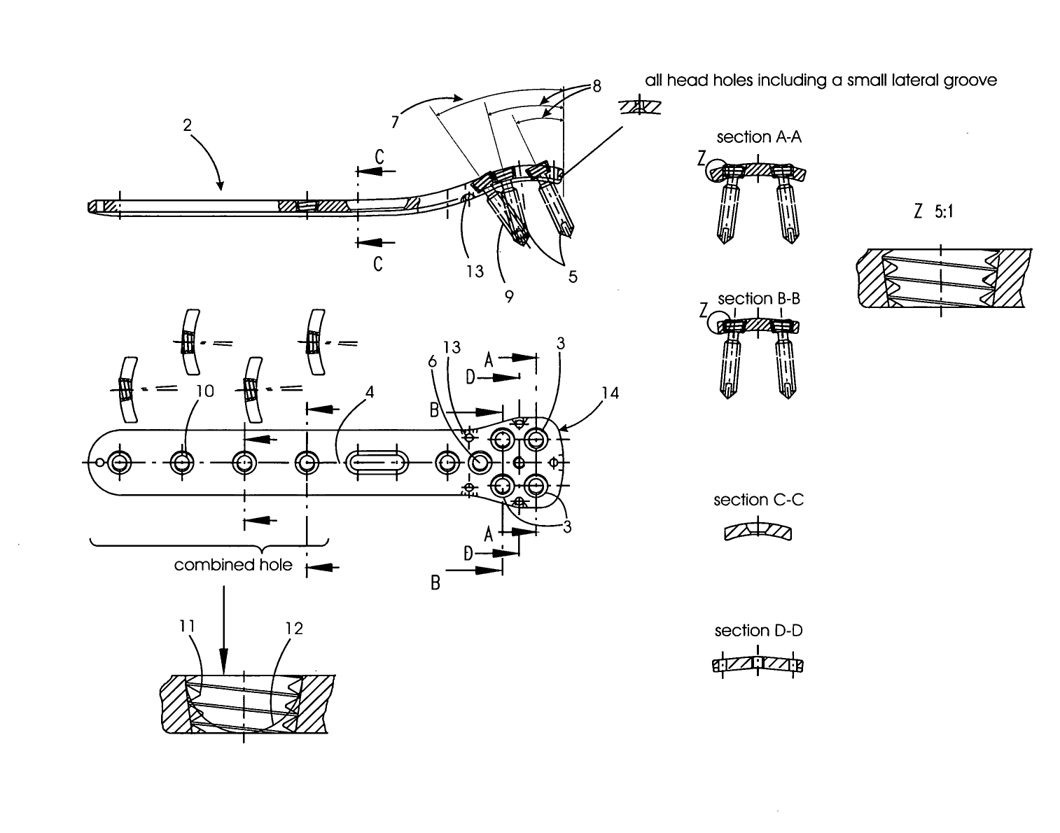

The invention relates to a small-fragment humeral head plate which is used to fix, in a stable manner, bone fracture fragments to the humeral head and shaft near to the body, comprising an elongated plate part, which has an arched cross-section, and an anatomically preformed head part adjacent thereto, and bores which are arranged in the plate part and in the head part, said bores having a conical thread used to receive bone screws which comprise, respectively, a screw head with a complementary conical thread, such that an angularly stable position is produced upon screwing, according to the preamble of patent claim 1.

Osteosynthesis plates made of titanium or implant steel, also in an anatomically preformed shape, are prior art.

Such plates are adapted with respect to their longitudinal and cross-sectional dimensions to the respective necessary operative conditions and comprise various bores for receiving cortical screws or spongiosa screws in the head part and the plate part. The diameter of the bores is thereby adapted to the corresponding screws, whereby it is likewise known to incorporate a conical thread into the bores, which communicates with a conical mating thread of a cortical screw head, so that a screwed-in screw adopts an angularly stable position. With such angularly stable screw connections it is not imperative that the plate rests on the bone surface, so that the healing process is accelerated.

Angularly stable small-fragment humeral head plates have the object of fixing, in a stable manner, bone fragments to the humerus near the body. Especially if the humeral head is split into several fragments, the humeral head comprises several bores for receiving a corresponding number of fixing screws.

However, it has shown that, specifically if a plurality of screw holes are provided in the head part of such a plate, the stability of the plate suffers, which results in a danger of breakage particularly in the transition region between the head part and the plate part.

Based on the foregoing it is, therefore, the object of the invention to provide an advanced small-fragment humeral head plate which is used to fix, in a stable manner, bone fracture fragments to the humeral head and shaft near to the body, wherein the novel plate is to permit a safe fixation also of a larger number of fragments such that the overall stability of the plate does not suffer in an undesired manner and without rendering the operative handling more difficult.

The solution to the object of the invention is achieved with a small-fragment humeral head plate which is used to fix, in a stable manner, bone fracture fragments to the humeral head and shaft near to the body according to the combination of features defined in patent claim 1, with the dependent claims comprising at least useful embodiments and advancements.

According to the invention, a symmetrically distributed group of bores are embodied in the head part, wherein the axis of symmetry coincides with the longitudinal central axis of the plate.

The first, external bores on the head part are oriented in relation to each other at an angle of approximately 12° to 17°, preferably 15°.

The second, internal bores on the head part of said symmetrical group of bores are oriented in relation to each other at an angle which is smaller than the angle at which the external bores on the head part are oriented in relation to each other. This smaller angle preferably amounts to 10°.

The first and second bores of the symmetrically distributed group have an inclined position towards the head part exterior in relation to the perpendicular of the longitudinal axis of the plate.

According to the invention, an additional bore is preferably embodied on the longitudinal central axis at a predetermined distance in relation to the proximal end of the plate, the inclined position of which is greater than that of the first and second bores.

The aforementioned additional bore specifically permits a better fixation of an upper calvarium fragment.

The predetermined distance of the additional bore in relation to the proximal end is within the range between <25 mm and >10 mm, in an exemplary embodiment of an angularly stable humeral head plate at approximately 20 mm originating from the proximal plate end or plate edge, respectively.

The additional bore serves to accommodate an angularly stable head attachment screw in the range of a diameter of approximately 3.5 mm to 4 mm.

In relation to the perpendicular to the longitudinal axis of the plate the inclined position angle of the additional bore is substantially at 30° and 40°.

The inclined position angle of the additional bore can also be defined in relation to the longitudinal central axis in the range between approximately 55° and 75°, preferably at 60° to 70°.

The lateral angle position of the additional bore in relation to the plate upper surface is in the range between +10° and −10°, preferably at 0°.

The bores provided in the plate part are preferably positioned on the longitudinal central axis. At least some of these bores are embodied as a so-called combined hole, comprising a conical thread and a spherical countersink for receiving either angularly stable screws, but also screws having a spherical head.

Preferably, at least one smaller diameter bore including a small lateral groove is provided in the transitional edge region between the head part and the plate part. This bore, or several bores embodied in this way serve to receive needle and thread if the plate resting on the bone is sewn up.

In a preferred embodiment of the invention, a further smaller diameter threaded bore as well as a guide bore are provided in the head part so as to temporarily fix a massive target block.

The target block comprises first and second bores as well as an additional bore complementarily to those in the head part, so that the target block can be used as drilling template and screw guide.

The head part has, in combination with the screwed-in screws, slightly yielding, resilient properties, so that a certain dynamics is provided to enhance the healing process.

Because of the target block and its use as drilling template or drill guide the plate itself may be made of a thin material, which offers operative advantages.

The plate moreover includes bores known per se, used for fixing a Kirschner's wire, namely at the front and rear end of the plate.

The invention shall be explained in more detail below by means of an embodiment and with reference to a figure.

The figure shows a top view of an angularly stable humerus plate according to the embodiment. The figure moreover shows a lateral view including a partial section, and various sections along the lines of the top view and lateral view for illustrating specifically the angular positions of the bores.

The figure also shows the basic embodiment of the combined holes in the longitudinal part of the plate.

The humeral head plate according to the embodiment consists of a head part 1 and a plate part 2 in an elongated form.

A symmetrically distributed group of bores 3 is embodied in the head part, wherein the axis of symmetry coincides with the longitudinal central axis 4.

The first, external bores on the head part are embodied such that screws received therein are oriented in relation to each other at an angle of 12° to 17°, preferably 150. Reference be here made to the illustration according to section A-A.

The second, internal bores on the head part are oriented in relation to each other at an angle which is smaller than that of the external bores on the head part, preferably at 10°, which is shown by the illustration according to section B-B.

The first and the second bores have an inclined position towards the head part exterior in relation to the perpendicular of the longitudinal axis of the plate 4. This inclined position is recognizable by the orientation of the screws 5.

Moreover, an additional bore 6 is preferably embodied on the longitudinal central axis 4 and located at a predetermined distance in relation to the proximal end of the plate, the inclined position angle 7 of which deviates from the inclined position angle 8 of the first and second bores and is specifically greater than the inclined position angle of the first and second bores.

The predetermined distance of the additional bore 6 in relation to the proximal end of the plate is predetermined to be smaller than 25 mm.

The additional bore 6 serves to receive an angularly stable head attachment screw 9 analogously to the screws for the first and second bores.

As can be seen in the figure, the inclined position angle for the additional bore 6 in relation to the perpendicular to the longitudinal axis of the plate is substantially 30° to 45°, e.g. specifically 35°.

The corresponding inclined position angle 8 for the other bores in the head part can be derived from the dimensioning in the figure.

The bores 10 provided in the plate part 2, which are likewise positioned on the longitudinal central axis 4, are at least partially embodied as combined holes. This means that these bores comprise both a conical thread 11 and a deep-reaching spherical countersink 12, so that either an angularly stable screw or also a screw having a spherical head are usable.

Preferably, at least one further smaller diameter bore 13 including a small lateral groove is provided in the transitional edge region between the head part 1 and the plate part 2. This bore serves to receive needle and thread.

A further threaded bore 14 is provided in the head part 1, so as to temporarily fix a massive target block (not shown) on the head part 1. The target block includes first and second bores as well as an additional bore complementarily to those in the head part, so that the target block can be used as drilling template, that is, for guiding the drill, but also as an aid for introducing the screws. By this measure it is possible to embody the plate itself with a smaller thickness.

The head part 1 has, in connection with the screws used, yielding, slightly resilient properties, so that a certain dynamics is provided which promotes the healing process after a fracture.

1. Small-fragment humeral head plate which is used to fix, in a stable manner, bone fracture fragments to the humeral head and shaft near to the body, comprising an elongated plate part, which has an arched cross-section, and an anatomically preformed head part adjacent thereto, and bores which are arranged in the plate part and in the head part, said bores having a conical thread used to receive bone screws which comprise, respectively, a screw head with a complementary conical thread, such that an angularly stable position is produced upon screwing,
characterized in that
a symmetrically distributed group of bores are embodied in the head part, wherein the axis of symmetry coincides with the longitudinal central axis of the plate,
the first, external bores on the head part are oriented in relation to each other at an angle of approximately 12° to 17°, preferably 15°,
the second, internal bores on the head part are oriented in relation to each other at an angle which is smaller than that of the external bores on the head part, preferably at approximately 10°,
the first and second bores have an inclined position towards the head part exterior in relation to the perpendicular of the longitudinal axis of the plate, and
further an additional bore is preferably embodied on the longitudinal central axis at a predetermined distance in relation to the proximal end of the plate, the inclined position of which is greater than that of the first and second bores.

2. Plate according to claim 1,
characterized in that
the predetermined distance of the additional bore in relation to the proximal end of the plate is within the range between <25 mm and >10 mm.

3. Plate according to claim 2,
characterized in that
the additional bore is embodied in the diameter range between 3.5 mm and 4 mm for receiving an angularly stable head attachment screw.

4. Plate according to claim 1,
characterized in that
the inclined position angle of the additional bore in relation to the perpendicular to the longitudinal axis of the plate is substantially at 30° to 40°.

5. Plate according to claim 1,
characterized in that
the inclined position angle of the additional bore in relation to the longitudinal central axis is in the range between 55° and 75°, preferably between 60° to 70°.

6. Plate according to claim 1,
characterized in that
the lateral angle position of the additional bore in relation to the plate upper surface is in the range between +10° and −10°, preferably at 0°.

7. Plate according to claim 1,
characterized in that
the bores provided in the plate part are positioned on the longitudinal central axis and that at least some of these bores are embodied as a combined hole, comprising a conical thread and a spherical countersink for receiving either angularly stable screws or screws having a spherical head.

8. Plate according to claim 1,
characterized in that
at least one smaller diameter bore including a small lateral groove is preferably provided in the transitional edge region between the head part and the plate part.

9. Plate according to claim 1,
characterized in that
a further threaded bore as well as at least one guide bore are provided in the head part so as to temporarily fix a massive target block, wherein the target block comprises first and second bores as well as an additional bore complementarily to those in the head part, so that the target block can be used as drilling template and screw guide.

10. Plate according to claim 1,
characterized in that
the head part has, in combination with the screws, slightly yielding, resilient properties.

11. Plate according to claim 2,
characterized in that
the inclined position angle of the additional bore in relation to the perpendicular to the longitudinal axis of the plate is substantially at 30° to 40°.

12. Plate according to claim 3,
characterized in that
the inclined position angle of the additional bore in relation to the perpendicular to the longitudinal axis of the plate is substantially at 30° to 40°.

13. Plate according to claim 2,
characterized in that
the inclined position angle of the additional bore in relation to the longitudinal central axis is in the range between 55° and 75°, preferably between 60° to 70°.

14. Plate according to claim 3,
characterized in that
the inclined position angle of the additional bore in relation to the longitudinal central axis is in the range between 55° and 75°, preferably between 60° to 70°.

15. Plate according to claim 2,
characterized in that
the lateral angle position of the additional bore in relation to the plate upper surface is in the range between +10° and −10°, preferably at 0°.

16. Plate according to claim 3,
characterized in that
the lateral angle position of the additional bore in relation to the plate upper surface is in the range between +10° and −10°, preferably at 0°.

17. Plate according to claim 4,
characterized in that
the lateral angle position of the additional bore in relation to the plate upper surface is in the range between +10° and −10°, preferably at 0°.

18. Plate according to claim 5,
characterized in that
the lateral angle position of the additional bore in relation to the plate upper surface is in the range between +10° and −10°, preferably at 0°.

19. Plate according to claim 2,
characterized in that
the bores provided in the plate part are positioned on the longitudinal central axis and that at least some of these bores are embodied as a combined hole, comprising a conical thread and a spherical countersink for receiving either angularly stable screws or screws having a spherical head.

20. Plate according to claim 3,
characterized in that
the bores provided in the plate part are positioned on the longitudinal central axis and that at least some of these bores are embodied as a combined hole, comprising a conical thread and a spherical countersink for receiving either angularly stable screws or screws having a spherical head.
